(19) United States
(12) Patent Application Publication
(10) Pub. No.: US 2019/0011442 A1
(43) Pub. Date: Jan. 10, 2019

(54) METHOD FOR DETECTING SUBSTANCE OF INTEREST, METHOD FOR QUANTIFYING SUBSTANCE OF INTEREST, KIT, AND METHOD FOR PREPARING REAGENT

(71) Applicants: Ortho-Clinical Diagnostics Kabushiki Kaisha, Tokyo (JP); Furukawa Electric Co., Ltd., Tokyo (JP)

(72) Inventors: Masayuki Fukushima, Tokyo (JP); Masataka Nishida, Tokyo (JP); Michio Ohkubo, Tokyo (JP); Kazutomi Miyoshi, Tokyo (JP)

(21) Appl. No.: 16/066,639

(22) PCT Filed: Dec. 28, 2015
(86) PCT No.: PCT/JP2015/086567

§ 371 Date: Jun. 27, 2018

(51) Int. Cl. G01N 33/543

(52) U.S. Cl. CPC G01N 33/54326; G01N 33/5306; G01N 33/54393

(57) ABSTRACT

The purpose of the present invention is to provide: a method for detecting a substance of interest and a method for quantifying a substance of interest, whereby it becomes possible to detect or quantify the substance of interest properly using a reagent that can be produced with high yield; a kit; and a method for preparing a regent. A method for detecting a substance of interest in a sample comprises a step of mixing a first conjugate 10, a second conjugate 20 and the sample together, then placing the resultant mixture under conditions such that a stimulus-responsive substance 11 can agglomerate, and then determining the presence or absence of the occurrence of dispersion of the stimulus-responsive substance 11 or the occurrence of an event associating with the aforementioned dispersion, wherein the first conjugate 10 is a conjugate of a first substance that contains the stimulus-responsive substance 11 with a first affinity substance 13 for a substance of interest 50, and the second conjugate 20 is a conjugate of a second substance 21 that has a hydrophilic moiety or an electrically charged moiety with a second affinity substance 23 for the substance of interest 50. The second substance 21 contains particles having a specific gravity of 1.4 or more, and the first affinity substance 13 and the second affinity substance 23 can bond to the substance of interest 50 simultaneously at different sites in the substance of interest 50 from each other.

### TECHNICAL FIELD

The present invention relates to a method of detecting a detection target and a method of quantifying a detection target, a kit, and a method of preparing a reagent.

### BACKGROUND ART

Conventionally, the latex agglutination method is used to detect a detection target in a specimen. When an antigen is to be detected in a fluid such as a biological sample, the latex agglutination method involves mixing a latex with the fluid to measure the degree of agglutination, the latex carrying an antibody or a fragment thereof which specifically binds to the antigen, thereby detecting and quantifying the antigen (for example, see Patent Document 1).

According to the latex agglutination method, the presence of an antigen in a specimen results in cross-linking of a plurality of latex-conjugated antibodies, promoting agglutination of latex. The procedures as simple as described above can allow easy and fast detecting of an antigen. However, when the amount of an antigen is very small, cross-linking may not sufficiently be formed, resulting in poor agglutination of latex. For this reason, detection of an antigen which is present in a trace amount is difficult.

Accordingly, methods based on an enzyme-substrate reaction are also widely used, such as the ELISA method and the CLEIA method. According to these methods, for example, a primary antibody which can specifically bind to an antigen is allowed to bind to the antigen, and a secondary antibody having an enzyme is allowed to bind to the primary antibody. Then, a substrate for the enzyme is added, and the degree of an enzyme-catalyzed reaction is measured to detect or quantify the antigen.

However, these methods based on enzyme-substrate reactions inevitably require many special reagents such as secondary antibodies and luminescent reagents, resulting in high operating cost. Further, a measuring step should be ended in a very short time to prevent fading of light from a luminescent reagent (bleaching phenomenon). Therefore, there is a concern about insufficient measurement accuracy. Moreover, the above methods include multi-steps such as a step of incubating a sample and reagents, a step of washing the system, and a step of measuring luminescence, resulting in complicated operations. Furthermore, each step is very time consuming, and not practical for large scale processes.

In view of the above, the present inventors previously developed a technology for detecting and quantifying a detection target using a first conjugate in which a substance including a stimuli-responsive polymer is bound to an antibody against the detection target; and a second conjugate in which an electrically-charged or hydrophilic substance is bound to another antibody against the target (see Patent Documents 2 and 3). In the above technology, a mixture of the above two conjugates and a specimen is exposed to conditions where the stimuli-responsive polymer aggregates. Then, a decreased degree of aggregation of the stimuli-responsive polymer as determined by turbidimetry and the like indicates the presence of the antigen in the specimen.

According to the above technology, detection can be achieved only using a substance including a stimuli-responsive polymer, antibodies, and an electrically-charged or hydrophilic substance without particularly using any special reagent. This enables inexpensive and simple detection. Further, the above technology only requires measurement of the degree of aggregation inhibition, and does not rely on a system requiring an enzyme-catalyzed reaction. Therefore, operations can be performed rapidly.

Patent Document 1: Japanese Examined Patent Application Publication No. S58-11575

Patent Document 2: Pamphlet of PCT International Publication No. WO2008/001868

Patent Document 3: Pamphlet of PCT International Publication No. WO2009/084595

### DISCLOSURE OF THE INVENTION

### Problems to be Solved by the Invention

Meanwhile, the second conjugate used in Patent Document 2 needs to be separated from unbound antibody after a reaction in which an electrically-charged or hydrophilic substance is allowed to bind to antibody in the manufacturing process of the second conjugate. This is because the unbound antibody may deteriorate the detection/quantification sensitivity. However, unbound antibody (“Free Antibody” in FIG. 7c) and a second conjugate (“PEG-labeled Antibody” in FIG. 7c) were often found to share similar molecule weight and the like to each other. If that is the case, significant portions corresponding to the unbound antibody and the second conjugate may overlap in a chromatogram and the like. These portions can not be recovered. Therefore, the yield of the second conjugate is in a trade-off relationship with the detection/quantification sensitivity for a detection target when that second conjugate is used.

The present invention is made in view of the above actual circumstances. An object of the present invention is to provide a method of detecting a detection target and a method of quantifying a detection target, the methods being capable of appropriately detecting or quantifying the detection targets using a reagent which can readily be obtained in a high yield; a kit; and a method of preparing the reagent.

### Means for Solving the Problems

The present inventors found herein the following. Use of a substance including a particle having a relatively large specific gravity as an electrically-charged or hydrophilic substance (hereinafter referred to as the second substance) of the second conjugate enables use of a particle having a small diameter for the second conjugate. This can allow particles having large specific surface areas to be present more abundantly, leading to an improved specific activity. Further, the second conjugate can easily be recovered from a second affinity substance in a free form in a high yield by solid-liquid separation and the like. Moreover, use of the above second conjugate does not prevent the stimuli-responsive substance from experiencing aggregation inhibition when a detection target is present as long as the second substance has a hydrophilic or electrically-charged moiety. Then the present invention has been completed. Specifically, the present invention can provide the following.

(1) A method of detecting a detection target in a specimen, the method including the steps of:

mixing a first conjugate in which a first substance including a stimuli-responsive substance is bound to a first affinity substance having an affinity for the detection target, a second conjugate in which a second substance having a hydrophilic or electrically-charged moiety is bound to a second affinity substance having an affinity for the detection target, and the specimen; exposing the resulting mixture to conditions where the stimuli-responsive substance aggregates; and determining whether dispersion of the stimuli-responsive substance or an event having a correlation therewith is present,

the second substance including a particle having a specific gravity of 1.4 or more,

the first affinity substance and the second affinity substance being capable of simultaneously binding to the detection target at different sites of the detection target.

(2) A method of quantifying a detection target in a specimen, the method including:

mixing a first conjugate in which a first substance including a stimuli-responsive substance is bound to a first affinity substance having an affinity for the detection target, a second conjugate in which a second substance having a hydrophilic or electrically-charged moiety is bound to a second affinity substance having an affinity for the detection target, and the specimen; exposing the resulting mixture to predetermined conditions where the stimuli-responsive substance aggregates;

measuring the turbidity of the mixture or a parameter having a correlation therewith; and calculating the amount of the detection target in the specimen based on a correlation equation for relating the amount of the detection target with the turbidity or the parameter under the predetermined conditions,

the second substance including a particle having a specific gravity of 1.4 or more, the first affinity substance and the second affinity substance being capable of simultaneously binding to the detection target at different sites of the detection target.

(3) The method according to (1) or (2), in which the first substance includes a particulate magnetic substance, and

the method further includes applying magnetic force to the mixture after exposing the mixture to the conditions to separate the magnetic substance in an aggregated state.

(4) The method according to any one of (1) to (3), in which the second substance is configured such that a water soluble substance is attached to a surface of the particle.

(5) A method of preparing a reagent for use in the method according to any one of (1) to (4), the reagent including the second substance, the method including the steps of:

allowing the second substance to bind with the second affinity substance in water, and then performing solid-liquid separation to recover the second conjugate in a solid phase.

(6) A kit for detecting and/or quantifying a detection target, the kit including:

a first conjugate in which a first substance including a stimuli-responsive substance is bound to a first affinity substance having an affinity for the detection target, and a second conjugate in which a second substance having a hydrophilic or electrically-charged moiety is bound to a second affinity substance having an affinity for the detection target,

the second substance including a particle having a specific gravity of 1.4 or more.

(7) The kit according to (6), in which the first substance includes a particulate magnetic substance.

(8) The kit according to (6) or (7), in which the second substance is configured such that a water soluble substance is attached to a surface of the particle.

### Effects of the Invention

According to the present invention, by using the second conjugate, the stimuli-responsive substance can experience aggregation inhibition by virtue of the hydrophilic or electrically-charged moiety of the second substance when a detection target is present. This enables appropriate detection and quantification of the detection target. Further, use of a substance including a particle having a relatively large specific gravity as the second substance can improve specific activity, enhance sensitivity, and increase reaction efficiency. Moreover, the aforementioned second conjugate can easily be recovered from the second affinity substance in a free form in a high yield by solid-liquid separation and the like.

### BRIEF DESCRIPTION OF THE DRAWINGS

FIG. 1 shows a schematic configuration diagram of conjugates for use in an embodiment of the present invention.

FIG. 2 shows schematic diagrams illustrating a state in which the conjugates according to the above embodiment are used.

FIG. 3 shows a schematic configuration diagram of conjugates which were used in a method of detecting an autoantibody as a detection target according to an embodiment of the present invention (see Example).

FIG. 4 shows a mode of applying magnetic force in a method according to one Example of the present invention.

FIG. 5 shows measurement results of turbidity from the method according to one Example of the present invention.

FIG. 6 shows a correlation equation for relating the concentration of an antigen with turbidity in the method according to one Example of the present invention.

FIG. 7 shows chromatograms when a conjugate according to Conventional Example is separated.

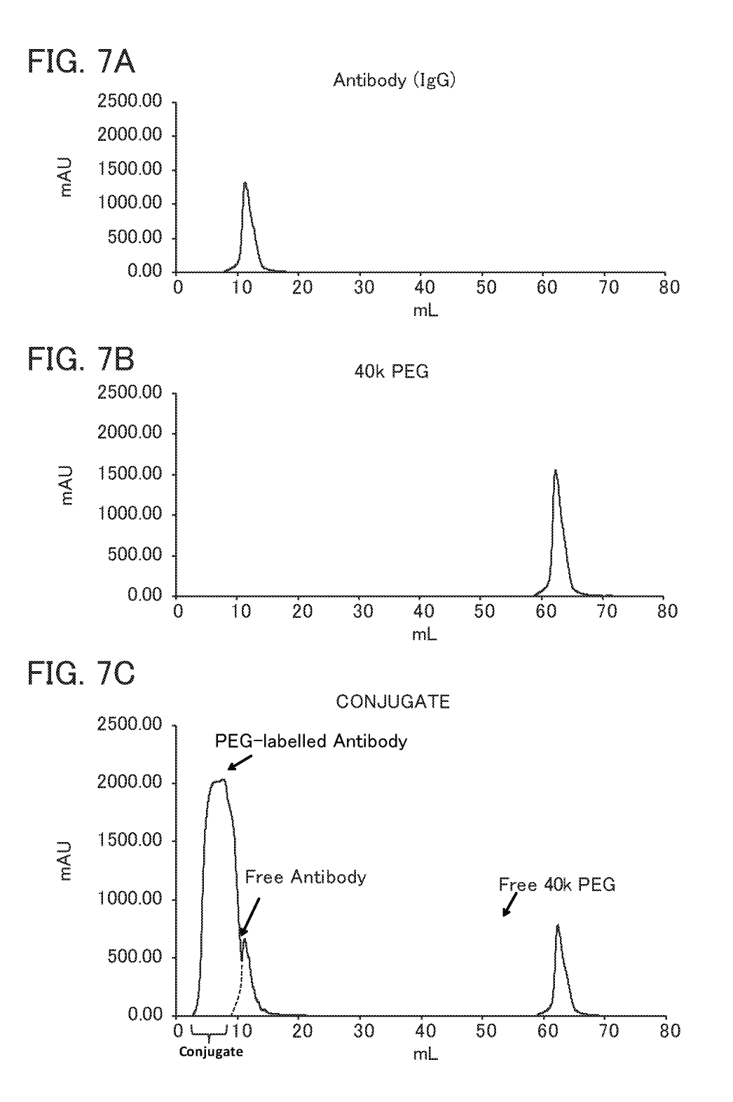

### PREFERRED MODE FOR CARRYING OUT THE INVENTION

Below, an embodiment of the present invention will be described with reference to the drawings.

Kit

A kit according to an embodiment of the present invention is intended for detecting or quantifying a detection target, and includes a first conjugate and a second conjugate. Below, each component will be described in detail.

First Conjugate

The first conjugate is configured such that a first substance including a stimuli-responsive polymer is bound to a first affinity substance having an affinity for a detection target.

First Substance

The first substance which may be used in an embodiment of the present invention includes a stimuli-responsive substance, and the stimuli-responsive substance can undergo a structural change in response to an external stimulus to control aggregation and dispersion. There is no particular limitation for the stimulus, and examples thereof include a change in temperature, irradiation of light, addition of an acid or a base (a change in pH), a change in an electric field, and the like.

In an embodiment of the present invention, the stimuli-responsive substance is preferably a temperature-responsive polymer capable of undergoing aggregation and dispersion in response to a change in temperature. It is noted that temperature-responsive polymers include a polymer having a lower critical solution temperature (hereinafter may also be referred to as LCST), and a polymer having an upper critical solution temperature (hereinafter may also be referred to as UCST).

Examples of a polymer having a lower critical solution temperature which may be used in the present invention include polymers of N-substituted (meth)acrylamide derivatives such as N-n-propylacrylamide, N-isopropylacrylamide, N-ethylacrylamide, N,N-dimethylacrylamide, N-acryloylpyrrolidine, N-acryloylpiperidine, N-acryloylmorpholine, N-n-propylmethacrylamide, N-isopropylmethacrylamide, N-ethylmethacrylamide, N,N-dimethylmethacrylamide, N-methacryloylpyrrolidine, N-methacryloylpiperidine, and N-methacryloylmorpholine; polyoxyethylene alkylamine derivatives such as hydroxypropylcellulose, partially acetylated polyvinyl alcohol, polyvinyl methyl ether, (polyoxyethylene-polyoxypropylene) block copolymer, and polyoxyethylene lauryl amine; polyoxyethylene sorbitan ester derivatives such as polyoxyethylene sorbitan laurate; (polyoxyethylene alkylphenyl ether) (meth)acrylates such as (polyoxyethylene nonylphenyl ether) acrylate and (polyoxyethylene octylphenyl ether) methacrylate; and polyoxyethylene (meth)acrylic acid ester derivatives, such as (polyoxyethylene alkyl ether) (meth)acrylates such as (polyoxyethylene lauryl ether) acrylate and (polyoxyethylene oleyl ether) methacrylate. Further, polymers thereof and copolymers of at least two of these monomers can also be used. Moreover, a copolymer of N-isopropylacrylamide and N-t-butylacrylamide can also be used. When a polymer including a (meth)acrylamide derivative is used, a different copolymerizable monomer may be copolymerized with that polymer in a range where the resulting product has a lower critical solution temperature. Among these, the following may be preferably used in the present invention: a polymer of at least one monomer selected from the group consisting of N-n-propylacrylamide, N-isopropylacrylamide, N-ethylacrylamide, N,N-dimethylacrylamide, N-acryloylpyrrolidine, N-acryloylpiperidine, N-acryloylmorpholine, N-n-propylmethacrylamide, N-isopropylmethacrylamide, N-ethylmethacrylamide, N,N-dimethylmethacrylamide, N-methacryloylpyrrolidine, N-methacryloylpiperidine, and N-methacryloylmorpholine; or a copolymer of N-isopropylacrylamide and N-t-butylacrylamide.

As a polymer having an upper critical solution temperature which can be used in the present invention, a polymer of at least one monomer selected from the group consisting of acryloylglycinamide, acryloylnipecotamide, acryloylasparaginamide, acryloylglutamineamide, and the like can be used. Moreover, a copolymer of at least two of these monomers may also be used. A different copolymerizable monomer such as acrylamide, acetylacrylamide, biotinol acrylate, N-biotinyl-N′-methacryloyl trimethylene amide, acryloyl sarcosine amide, methacryl sarcosine amide, acryloyl methyl uracil may be copolymerized with these polymers in a range where the resulting product has an upper critical solution temperature.

A substance such as a pH-responsive polymer capable of undergoing aggregation and dispersion in response to a pH change may also be used as a stimuli-responsive substance an embodiment of in the present invention. There is no particular limitation for the value of pH where a pH-responsive polymer undergoes a structural change, but it is preferably pH 4 to 10, more preferably pH 5 to 9 in view of preventing deteriorated detection/quantification accuracy which may be caused by denaturalization of the first conjugate, the second conjugate, and a specimen upon applying a stimulus.

For such a pH-responsive polymer, polymers having a group such as carboxyl, phosphoric acid, sulfonyl, and amino as a functional group can be exemplified. More specifically, those may be used in which monomers having dissociable groups such as (meth)acrylic acid, maleic acid, styrenesulfonic acid, 2-acrylamido-2-methylpropanesulfonic acid, phosphorylethyl (meth)acrylate, aminoethyl methacrylate, aminopropyl (meth)acrylamide, and dimethylaminopropyl (meth)acrylamide may be polymerized. Or those may be used in which monomers having these dissociable groups may be copolymerized with other vinyl monomers, for example, (meth)acrylic acid esters such as methyl (meth)acrylate, ethyl (meth)acrylate, butyl (meth)acrylate; vinyl esters such as vinyl acetate and vinyl propionate; vinyl compounds such as styrene, vinyl chloride, and N-vinyl pyrrolidone; (meth)acrylamides; and the like as long as pH-responsiveness is not impaired.

Particulate Magnetic Substance

The particulate magnetic substance used herein may be configured so as to include polyhydric alcohol and magnetite. There is no particular limitation for the polyhydric alcohol as long as it has at least two hydroxy groups in a constituent unit, and has an alcohol structure capable of binding to an iron ion. Examples thereof include, for example, dextran, polyvinyl alcohol, mannitol, sorbitol, and cyclodextrin. For example, Japanese Unexamined Patent Application, Publication No. 2005-82538 discloses a method of manufacturing a particulate magnetic substance using dextran. Further, an epoxy-containing compound which can form a polyhydric alcohol structure after ring opening, such as a glycidyl methacrylate polymer, can be used. The particulate magnetic substance (magnetic microparticles) prepared using such a polyhydric alcohol preferably has a mean particle diameter of 0.9 nm or more and less than 1000 nm so as to have good dispersibility. In particular, the mean particle diameter is preferably 2.9 nm or more and less than 200 nm in order to increase detection sensitivity for a detection target of interest.

According to an embodiment of the present invention, a relatively large specific surface area can be obtained when the second substance includes a particle having a relatively large specific gravity. Therefore, the aggregation inhibition effect by virtue of a hydrophilic or electrically-charged moiety tends to be maintained.

First Affinity Substance

The first affinity substance may be any substance capable of binding to a detection target, for example, an antibody capable of recognizing a detection target. The antibody as used herein may be any type of immunoglobulin molecules, or may be a fragment of an immunoglobulin molecule having an antigen binding site such as Fab. The antibody may also be a monoclonal antibody or a polyclonal antibody. Further, in another aspect, when the detection target is an antibody (immunoglobulin), for example, human immunoglobulin G, M, A, or E, the first affinity substance may include an antigen or an antigenic determinant which can be recognized by the respective human immunoglobulin G, M, A, or E, and in particular, may typically include an antigen or an antigenic determinant which can be recognized by an autoantibody. The term “antigenic determinant” as used herein does not necessarily have the exactly same structure as the corresponding naturally occurring antigen molecule, but may simply be a substance which can be recognized by an antibody as a detection target. It is noted that in order to clarify an aspect of the first affinity substance, an example is presented in the Example section of the present specification where an autoantibody (for example, an antibody which can recognize an anti-cyclic citrullinated peptide) is used as a detection target, and an autoantigen (for example, CCP; cyclic citrullinated peptide) is used as the first affinity substance.

Production of First Conjugate

The first conjugate may be produced by allowing the first substance to bind to the first affinity substance. There is no particular limitation for the binding method, but the method may include, for example, attaching one of a pair of substances having a mutual affinity (for example, avidin and biotin or glutathione and glutathione S-transferase) to the side of a first substance (for example, a portion of a stimuli-responsive substance) and attaching the other to the side of a first affinity substance (for example, a first antibody), allowing the first substance to bind to the first affinity substance through these substances having a mutual affinity.

Specifically, as described in WO01/009141, attachment of biotin to a stimuli-responsive substance may be performed by attaching biotin or the like to a polymerizable functional group such as a methacrylic group and an acrylic group to obtain an addition-polymerizable monomer, which is then copolymerized with another monomer. Meanwhile, attachment of avidin or the like to a first affinity substance may be performed in accordance with a conventional method. Next, when the biotin-attached stimuli-responsive substance is mixed with the avidin-attached first affinity substance, the first affinity substance and the stimuli-responsive substance polymer are bound together through binding of avidin and biotin.

Alternatively, a method may be used including: copolymerizing a monomer having a functional group such as carboxyl, amino, or epoxy with another monomer when a polymer is manufactured; and attaching a substance having an affinity for antibody (for example, Melon® gel, protein A, protein G) to the resulting polymer through the functional group in accordance with a method well known in the art. The substance having an affinity for antibody obtained as described above is allowed to bind to a first antibody to prepare a first conjugate in which the stimuli-responsive substance is bound to the first antibody having an affinity for an antigen as a detection target.

Alternatively, a monomer having a functional group such as carboxyl, amino, or epoxy may be copolymerized with another monomer when a polymer is manufactured, and a first antibody having an affinity for an antigen as a detection target may be attached directly to the functional group in accordance with a conventional method.

Alternatively, the first affinity substance and the stimuli-responsive substance may be allowed to bind to a particulate magnetic substance.

Centrifugal separation may be performed to purify the first conjugate after the first affinity substance is exposed to conditions where the stimuli-responsive polymer aggregates. The first conjugate may be purified by a method including attaching a particulate magnetic substance to a stimuli-responsive polymer, and further attaching a first affinity substance to the particulate magnetic substance, then exposing these to conditions where the stimuli-responsive polymer aggregates, and applying magnetic force to collect the particulate magnetic substance.

A particulate magnetic substance may be attached to a stimuli-responsive polymer by a method well known in the art such as a method in which attachment is mediated via a reactive functional group; a method in which a polymerizable unsaturated bond is introduced to an active hydrogen on polyhydric alcohol or polyhydric alcohol of a magnetic substance, and graft polymerization is performed; and the like (for example, see ADV. Polym. Sci., vol. 4, p 111, 1965 and J. Polymer Sci., Part-A, 3, p 1031, 1965).

Second Conjugate

The second conjugate is configured such that a second substance having a hydrophilic or electrically-charged moiety is bound to a second affinity substance having an affinity for a detection target. In particular, the second substance for use in an embodiment of the present invention includes a particle having a relatively large specific gravity. This can improve specific activity, and also enables the second conjugate to be easily recovered from the second affinity substance in a free form in a high yield by solid-liquid separation and the like. Moreover, use of the above second bound substance does not prevent the stimuli-responsive substance from experiencing aggregation inhibition due to the presence of the hydrophilic or electrically-charged moiety of the second substance when a detection target is present. This enables appropriate detection and quantification of a detection target to be performed.

The second substance should include a particle having a specific gravity larger than that of water (hereinafter, a particle having a high specific gravity included in the second substance may be referred to as a “high-specific gravity particle.”) in view of easily achievable solid-liquid separation. The specific gravity of a high-specific gravity particle is preferably 1.4 or more, more preferably 1.6 or more, even more preferably 1.7 or more, yet even more preferably 1.8 or more, and in particular preferably 1.9 or more. The upper limit of the specific gravity is more preferably 2.5, even more preferably 2.3, yet even more preferably 2.2, and in particular preferably 2.1. As used herein, the specific gravity refers to a value as measured in accordance with a method defined in JIS Z 8807.

A particle of the second substance having a specific gravity falling within the above ranges will have a relatively small particle diameter. Therefore, a particle having a large specific surface area per particle can be used. Further, a larger number of particles can be allowed to be present, leading to an increased specific surface area of the overall population of particles. This in turn can improve specific activity, enhance sensitivity, and also increase reaction efficiency. A particle of the second substance having a specific gravity falling within the above ranges can show good separability, and thus can be sedimented even at centrifugal force (gravitational acceleration) where a conventional low-specific gravity particle can not be sedimented by centrifugation.

It is noted that a particle of the second substance preferably has a relatively small scattering intensity for the light at a wavelength used in turbidimetry described below. A particle of the second substance having a relatively large scattering intensity is not preferred because the baseline of measured values of turbidity increases as the concentration of the particle increases.

There is no particular limitation for the high-specific gravity particle of the second substance, but examples thereof include, for example, particles of silica, acrylic resin, or metal because they tend to have both of the aforementioned properties: a relatively large specific gravity and a relatively small scattering intensity. Among these, particles of silica or acrylic resin are preferred, and particles of silica (hereinafter may also be referred to as “silica particles”) are more preferred. The silica particle used in the present invention may be a particle including silicon dioxide as the main component, or may be a particle of so-called silica, including a particle of quartz or rock crystal. Silica particles, which are highly hydrophilic by virtue of silanol groups (Si—OH) present on the particle surfaces, are preferred because they can easily be reacted in water. Silica particles are also preferred because they are non-toxic, easy to handle, and easy to manufacture.

There is no particular limitation for other particles which can be used in combination with the aforementioned high-specific gravity particle, but they include, for example, compounds having electrically-charged moieties as described below and/or compounds having hydrophilic moieties. For example, they may be latex particles and the like.

The high-specific gravity particle included in the second substance may have a mean particle diameter of 0.001 to 0.50 μm or 0.005 to 0.20 μm. In particular, high-specific gravity particles can have a relatively small mean particle diameter falling within the above ranges. As used herein, the term “mean particle diameter” refers to a value obtained by determining an occupied area of particles using an image processing device based on the total projected area of 50 particles randomly selected from an image of transmission electron microscopy (TEM), scanning electron microscopy (SEM), or the like, and calculating the mean value of the diameters of circles (the diameter of the mean circle), which corresponds to a value obtained by dividing the occupied area of particles by the number (50) of selected particles. The above mean particle diameter does not encompass the particle diameter of a secondary particle in which primary particles are aggregated.

The high-specific gravity particle included in the second substance may have a specific surface area of 10 to 900 m²/g, more preferably 50 to 500 m²/g, and more preferably 100 to 300 m²/g. In particular, the high-specific gravity particle can have a relatively large specific surface area falling within the above ranges. As used herein, the specific surface area refers to a value as measured in accordance with a method defined in JIS Z 8830.

The second substance may have a functional group and the like for attaching a second affinity substance to the surface of a high-specific gravity particle, or the inside or termini of a polymer chain, and may have a hydrophobic group for enabling physical adsorption of a second affinity substance. In particular, the second substance preferably has a hydrophobic group for enabling physical adsorption of a second affinity substance in view of easy and rapid manufacture of the second conjugate. The hydrophobic group will be masked by the adsorbed second affinity substance. Therefore, the aggregation inhibition by virtue of the hydrophilic or electrically-charged moiety will not be significantly interfered with. For example, when a silica particle is used as the high-specific gravity particle, the functional group and the like for attaching a second affinity substance may be a silanol group on the surface of the silica particle.

When the high-specific gravity particle itself has a hydrophilic or electrically-charged moiety, the second substance having a hydrophilic or electrically-charged moiety may consist of the high-specific gravity particle alone, or may be configured such that the high-specific gravity particle is attached to a compound having a hydrophilic or electrically-charged moiety. For example, when a silica particle is used as the high-specific gravity particle, a silanol group present on the surface of the silica particle may serve as a hydrophilic group for the second substance having a hydrophilic or electrically-charged moiety, or for example, may be configured such that the silanol group is attached to a compound having a hydrophilic or electrically-charged moiety. Further, when the high-specific gravity particle itself does not have a hydrophilic or electrically-charged moiety, the second substance having a hydrophilic or electrically-charged moiety may be configured such that the high-specific gravity particle is attached to a compound having a hydrophilic or electrically-charged moiety.

For the second substance, a compound having an electrically-charged moiety other than a high-specific gravity particle is, for example, preferably a polyanion or a polycation. The term “polyanion” means a substance having a plurality of anion groups, and the term “polycation” means a substance having a plurality of cation groups. Polyanions include, for example, nucleic acids such as DNA and RNA. These nucleic acids have polyanionic properties because they each have a plurality of phosphodiester groups along the nucleic acid backbone. Further, polyanions also include polypeptides having a large number of carboxyls (polypeptides including amino acids such as glutamic acid and aspartic acid); polymers including polyacrylic acid, polymethacrylic acid, polysulfonic acid, and acrylic acid, and/or methacrylic acid as polymerizable components; polysaccharides such as carboxymethylcellulose, hyaluronic acid, and heparin; and the like. Polycations include, for example, polylysine, polyarginine, polyornithine, polyalkylamine, polyethyleneimine, polypropylethyleneimine, and the like. It is noted that the number of functional groups on a polyanion (carboxyl) or a polycation (amino) is preferably 25 or more. Further, they also include a latex particle (may include polystyrene and the like) having a carboxyl group; and the like.

Moreover, examples of a compound having a hydrophilic moiety other than the high-specific gravity particle for the second substance include, for example, polymers having ester bonds such as polyethylene glycol, polypropylene glycol, polyethylene oxide, and polypropylene oxide; polymers having alcoholic hydroxyl groups such as polyvinyl alcohol; polysaccharides such as dextran, cyclodextrin, agarose, and hydroxypropylcellulose; polypeptides including neutral amino acids; and the like.

As the compound having a hydrophilic moiety other than the high-specific gravity particle for the second substance, erythrocyte-derived cell membrane and the like may also be used besides the aforementioned polymer particles. Commercially available and inexpensive erythrocyte to which affinity substances (antibodies) are bound may be disrupted according to a conventional method to obtain membrane, which can be used as the erythrocyte-derived cell membrane.

When the particle diameter of the second substance is too large, the detection/quantification efficiency tends to be decreased. On the other hand, when it is too small, the efficiency of solid-liquid separation tends to be decreased. The mean particle diameter of the second substance may be appropriately selected in view of the above, and it may be, for example, 0.001 to 0.50 μm or 0.005 to 0.20 μm.

In an embodiment of the present invention, preferably used is a second substance in which a water soluble substance is attached on the surface of the high-specific gravity particle. The aggregation inhibition effect may be improved by virtue of the water soluble substance attached on the surface of the high-specific gravity particle. The water soluble substance which can be used herein may be the aforementioned substance having an electrically-charged moiety (a polyanion, a polycation), a water-soluble high-molecular weight compound, and the like. The attachment thereof may be either via chemical adsorption or via physical adsorption.

The second substance may be used as alone or in combination of two or more.

Second Affinity Substance

The second affinity substance can bind to the same detection target as the first affinity substance at a different site than the first affinity substance binds to. The first affinity substance and the second affinity substance may be, for example, antibodies, for example, monoclonal antibodies, which can recognize different antigenic determinants of a detection target.

Production Method

The second conjugate may be produced by allowing the second substance to directly or indirectly bind to the second affinity substance. There is no particular limitation for the binding method, but the method may include, for example, attaching one of a pair of substances having a mutual affinity (for example, avidin and biotin or glutathione and glutathione S-transferase) to the side of the second substance and attaching the other to the side of the second affinity substance (for example, a second antibody), allowing the second substance to indirectly bind with the second affinity substance through these substances having a mutual affinity.

For direct binding of the second substance to the second affinity substance, they may be bound together through a functional group. For example, when a functional group is used, binding may be performed in accordance with the maleimide-thiol coupling method by Ghosh et al. (Ghosh et al., Bioconjugate Chem., 1, 71-76, 1990). Specifically, the following two methods can be exemplified.

According to a first method, first, a mercapto group (may also be referred to as a sulfhydryl group) is introduced into the 5′ end of a nucleic acid while 6-maleimide hexanoic acid succinimide ester (for example, “EMCS (product name)” (Dojindo Laboratories)) is allowed to react with an antibody to introduce a maleimide group. Next, these two substances are allowed to be bound together through the mercapto group and the maleimide group.

According to the second method, first, a mercapto group is introduced into the 5′ end of a nucleic acid as in the first method, and the mercapto group is further allowed to react with N,N-1,2-phenylenedimaleimide as a homo-bifunctional reagent to introduce a maleimide group into the 5′ end of the nucleic acid while a mercapto group is introduced into an antibody. Next, these two substances are allowed to be bound together through the mercapto group and the maleimide group.

In addition, as a method of introducing a nucleic acid into a protein, methods described in, for example, Nucleic Acids Research, 15; 5275 (1987), and Nucleic Acids Research, 16; 3671 (1988) are known. These technologies may be used for binding of a nucleic acid to an antibody.

According to Nucleic Acids Research, 16: 3671 (1988), first, an oligonucleotide is allowed to react with cystamine, carbodiimide, and 1-methylimidazole to introduce a mercapto group into the hydroxy group of the 5′ end of the oligonucleotide. The oligonucleotide into which the mercapto group has been introduced is purified, and then reduced with dithiothreitol. Subsequently, 2,2′-dipyridyl disulfide is added to introduce a pyridyl group into the 5′ end of the oligonucleotide through a disulfide bond. Meanwhile, iminothialene is allowed to react with a protein to introduce a mercapto group. The oligonucleotide into which the pyridyldisulfide group has been introduced is mixed with the protein into which the mercapto group has been introduced to allow the pyridyl group to specifically react with the mercapto group, thereby attaching the protein to the oligonucleotide.

According to Nucleic Acids Research, 15: 5275 (1987), first, an amino group is introduced into the 3′ end of an oligonucleotide, and dithio-bis-propionic acid-N-hydroxysuccinimide ester (may be abbreviated as dithio-bis-propionyl-NHS) as a homo-bifunctional reagent is then allowed to react. After the reaction, the disulfide bond in the dithio-bis-propionyl-NHS molecule is reduced by adding dithiothreitol to introduce a mercapto group into the 3′ end of the oligonucleotide. For treating a protein, a hetero-bifunctional cross-linking agent may be used as described in Japanese Unexamined Patent Application, Publication No. H5-48100. First, the hetero-bifunctional cross-linking agent which has a first reactive group (succinimide) capable of reacting with a functional group (for example, an amino group) in a protein and a second reactive group (for example, maleimide and others) capable of reacting with a mercapto group is allowed to react with the protein to introduce a second reactive group into the protein. This will be used as a pre-activated protein reagent. This protein reagent obtained as described above is covalently bonded with the mercapto group of the thiolated polynucleotide.

Even when a polyanion or a polycation other than nucleic acid is used, the second conjugate can be produced in accordance with similar operations as described above by introducing a mercapto group into a terminus thereof.

The second conjugate is not limited to those having the aforementioned chemical bonds, but may be produced by physical adsorption of the second affinity substance to the second substance. This approach is advantageous in that the process can be completed rapidly and simply. It is noted that the second substance preferably has a hydrophobic moiety as described above in view of promoting physical adsorption.

Conditions for the aforementioned reaction steps may be appropriately selected in view of reaction efficiency, suppressed inactivation, and the like. There is no particular limitation for temperature, but it may be 10 to 50° C. or 20 to 40° C.

Regardless of which of the methods is used, the second affinity substance in a free form needs to be separated and removed from the second conjugate after the reaction. The residual second affinity substance in a free form may compete with the second conjugate and bind to a detection target, reducing the aggregation inhibition effect of the second conjugate.

Conventionally, the second conjugate is isolated and recovered by chromatography such as molecular weight fractionation. This approach is complicated, and also may result in a poor yield due to overlapping of the second conjugate and the second affinity substance in a chromatogram. In contrast, according to an embodiment of the present invention, the second conjugate including the second substance is present as the solid phase while the second affinity substance in a free form is present as the liquid phase. Therefore, the second conjugate can be recovered more easily and in a high yield by solid-liquid separation. It is noted that the second substance in a free form may be present along with the second conjugate, but the second substance in a free form does not compete with the second conjugate. Therefore, this is not a problem.

Solid-liquid separation may be performed in accordance with a conventional method, and a centrifugation and the like may be used. After the centrifugation, a washing step of removing a supernatant, re-dispersing a pellet, and performing centrifugation may be further performed. It is noted that the separation efficiency may be decreased when the centrifugal force upon centrifugation is too small. On the other hand, the time required to re-disperse a pellet tends to be prolonged when it is too large. In view of the above, the centrifugal force may be 5000 to 100000 g or 10000 to 30000 g although there is no particular limitation for the centrifugal force.

Detection Method

The detection method according to an embodiment of the present invention includes steps of first mixing a first conjugate, a second conjugate, and a specimen; exposing the resulting mixture to conditions where a stimuli-responsive substance aggregates; and determining whether dispersion of the stimuli-responsive substance or an event having a correlation therewith is present. Below, the procedures will be described in detail.

Mixing/Aggregation

First, the first conjugate is mixed with the second conjugate in a container, and the specimen is then added to obtain a mixture. Subsequently, the mixture is exposed to conditions where the stimuli-responsive polymer aggregates. Then, the stimuli-responsive substance experiences aggregation inhibition to remain dispersed by virtue of the hydrophilic moiety of the second conjugate when a detection target is present. In contrast, when the detection target is not present, the stimuli-responsive substance does not experience aggregation inhibition, and thus undergoes aggregation.

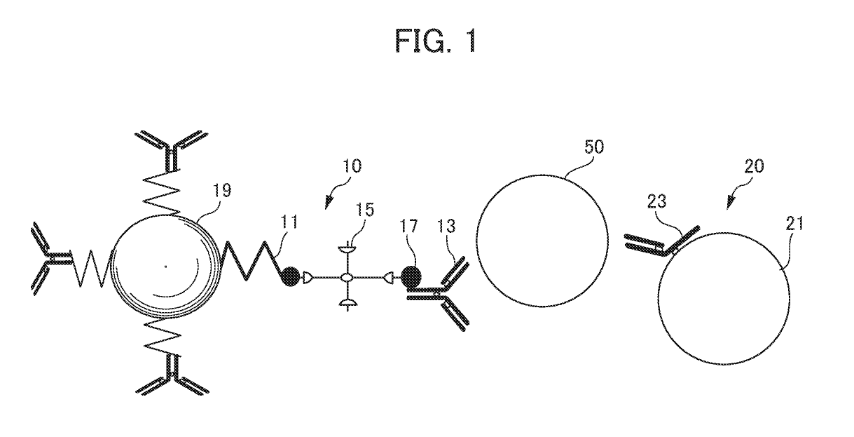

This phenomenon will be described with reference to FIGS. 1 to 3.

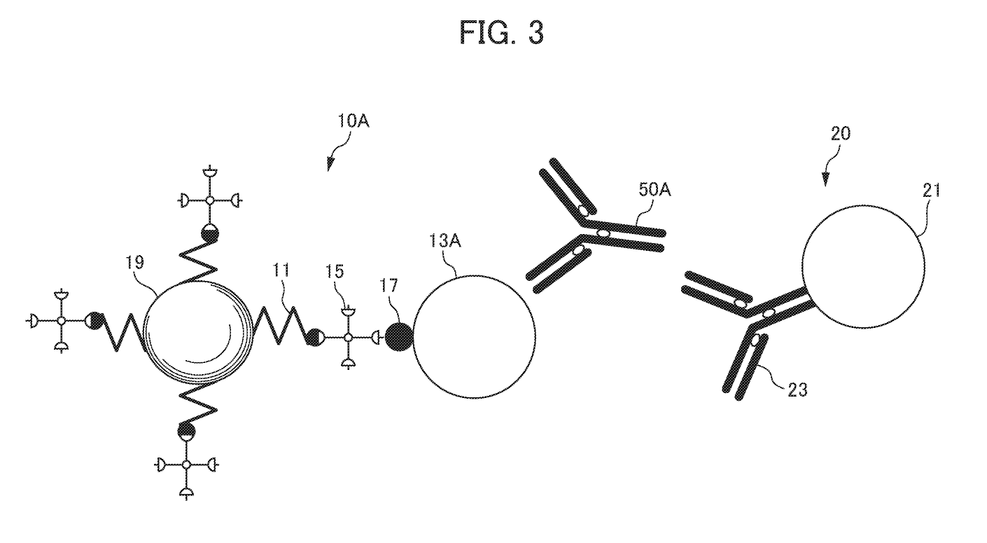

As shown in FIG. 1, a first conjugate 10 includes a stimuli-responsive polymer 11, and the stimuli-responsive polymer 11 is bound to a first antibody 13 against a detection target 50 through avidin 15 and biotin 17. (It is noted that in a configuration of FIG. 3, an autoantigen 13A is bound to a detection target 50A) The first conjugate 10 also includes a particulate magnetic substance 19, and the stimuli-responsive polymer 11 is attached to the surface of the magnetic substance 19. Meanwhile, a second conjugate 20 includes a second substance 21 having a hydrophilic moiety, and the second substance 21 is attached to a second antibody 23 against the detection target 50. Further, the first antibody 13 (in the case of FIG. 3, the autoantigen 13A capable of binding to the detection target 50A) and the second antibody 23 can simultaneously bind to the detection target 50 (in the case of FIG. 3, the autoantibody 50A) at different sites of the detection target 50 (in the case of FIG. 3, the autoantibody 50A).

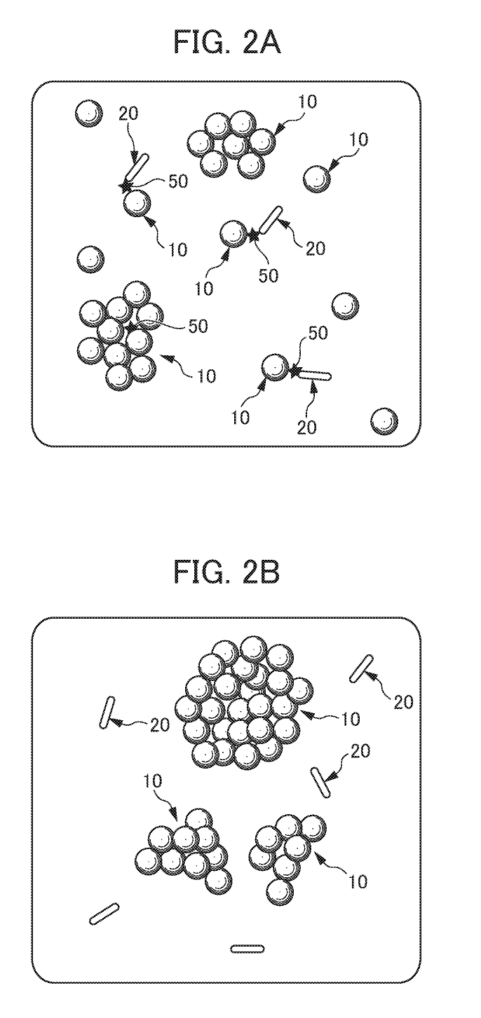

As shown in FIG. 2, when the detection target 50 is present, the stimuli-responsive polymer 11 experiences aggregation inhibition and remains dispersed by virtue of the hydrophilic moiety in the second conjugate 20 upon exposing the mixture of the first conjugate 10, the second conjugate 20 and a specimen to predetermined conditions (FIG. 2(A)). In contrast, when the detection target 50 (in the case of FIG. 3, the autoantibody 50A) is not present, the stimuli-responsive polymer 11 does not experience aggregation inhibition, and thus undergoes aggregation (FIG. 2(B)).

In order to allow the stimuli-responsive substance to aggregate, for example, when a temperature-responsive polymer is used, a container containing the liquid mixture may simply be transferred to an incubator at a temperature where the temperature-responsive polymer undergoes aggregation. For example, when a polymer having a lower critical solution temperature (LCST) of 32° C. is used, a container containing the liquid mixture may be transferred to an incubator at 32° C. or more to allow the temperature-responsive polymer to aggregate. Alternatively, when a polymer having an upper critical solution temperature (UCST) of 5° C. is used, a container containing the liquid mixture may be transferred to an incubator at less than 5° C. to allow the temperature-responsive polymer to aggregate.

Alternatively, when a pH-responsive polymer is used, an acid solution or an alkali solution may be added to a container containing the liquid mixture. Specifically, an acid solution or an alkali solution may simply be added to a container containing a dispersion liquid mixture having a pH outside the pH range where the pH-responsive polymer undergoes a structural change so that the pH of the content of the container is changed to a pH within the pH range where the pH-responsive polymer undergoes a structural change. For example, when a pH-responsive polymer undergoing aggregation at pH 5 or less and dispersion above pH 5 is used, an acid solution may simply be added to a container containing a liquid mixture dispersed above pH 5 so that the pH becomes 5 or less. Alternatively, when a pH-responsive polymer undergoing aggregation at pH 10 or more and dispersion below pH 10 is used, an alkali solution may simply be added to a container containing a liquid mixture dispersed below pH 10 so that the pH becomes 10 or more. There is no particular limitation for a pH where a pH-responsive polymer undergoes a structural change, but it is preferably pH 4 to 10, more preferably pH 5 to 9.

Alternatively, when a photo-responsive polymer is used, a container containing a liquid mixture may simply be irradiated with light having a wavelength capable of allowing the photo-responsive polymer to aggregate. A preferred type of light for initiating aggregation may vary depending on the type and structure of a photo-responsive functional group included in the photo-responsive polymer, but in general, ultraviolet or visible light having a wavelength of 190 to 800 nm may be used conveniently. In that case, the intensity is preferably 0.1 to 1000 mW/cm². It is noted that in view of increased measurement accuracy, a photo-responsive polymer preferably will not readily undergo dispersion, in other words, will remain aggregated upon irradiation with light for measuring the turbidity. When a photo-responsive polymer is used which undergoes dispersion upon irradiation with light for measuring the turbidity, a shortened irradiation time can increase measurement accuracy.

It is noted that aggregation of a temperature-responsive polymer may be initiated after or simultaneously in parallel with binding of the first conjugate and the second conjugate to a detection target, but the latter is preferred in view of a shortened treatment time. However, the former is preferred when the conditions where the temperature-responsive polymer aggregates significantly differ from the conditions where the first conjugate and the second conjugate bind to the detection target.

Here, the lower critical solution temperature may be determined as follows. First, a sample is transferred into a cell in an absorption spectrometer, and then heated at a rate of 1° C./minute. During this period, changes in transmittance at 550 nm are recorded. Here, a value of transmittance when a polymer is dissolved and transparent is considered as 100%, and a value of transmittance when the polymer is completely aggregated is considered as 0%. Then, a temperature at which transmittance is 50% is defined as LCST.

Further, the upper critical solution temperature may be determined as follows. A sample is cooled at a rate of 1° C./minute, and changes in transmittance at 550 nm are recorded in a similar manner. Here, a value of transmittance when a polymer is dissolved and transparent is considered as 100%, and a value of transmittance when the polymer is completely aggregated is considered as 0%. Then, a temperature at which transmittance is 50% is defined as UCST.

Determination

The presence of dispersion can be determined, for example, by visual inspection or by turbidimetry. Turbidity may be computed from light transmittance as measured with a light scattering instrument. Low turbidity means that the stimuli-responsive polymer experiences aggregation inhibition, suggesting the presence of a substance to be detected. Here, the wavelength of light used may be appropriately selected so that a desired detection sensitivity can be obtained depending on the particle diameter and the like of a magnetic substance. The wavelength of light is preferably in the range of visible light (for example, 550 nm) in view of availability of a common general-purpose instrument.

Visual inspection or turbidimetry may be performed at a predetermined time point either continuously or intermittently. Further, determination may be performed based on the difference in measured values of turbidity between one time point and another time point.

There is no particular limitation for dispersion of a stimuli-responsive substance or an event correlated therewith, but it may be a signal upon being developed with a developing carrier (thin layer chromatography); the degree of increase in a magnetic field when the first substance includes a magnetic substance; and the like.

A detection method based on a signal upon being developed with a developing carrier is disclosed in WO2010/137532. Specifically, the method includes the steps of: developing a mixture with a developing carrier, the mixture having been exposed to conditions where a stimuli-responsive substance aggregates, or exposing a mixture to conditions where the stimuli-responsive substance aggregates, the mixture having been in the course of development; detecting a signal resulting from the presence of a first conjugate or a second conjugate in the developing carrier; and determining whether the signal is different from a corresponding signal in the absence of a detection target, in which the signal different from the corresponding signal indicates that the detection target is present in a specimen. The above method uses a phenomenon in which an aggregated stimuli-responsive substance will slowly develop in an appropriately selected developing carrier.

A detection method based on the strength of a magnetic field is disclosed in WO2009/084596. Specifically, the method includes the steps of: exposing a mixture under conditions where a stimuli-responsive substance aggregates; then applying magnetic force to measure a magnetic field arising therefrom; and detecting a detection target based on the degree of increase in the magnetic field after applying the magnetic force. The above method uses a phenomenon in which aggregates will create a larger magnetic field.

Method of Quantification

The quantification method according to an embodiment of the present invention includes: mixing a first conjugate, a second conjugate, and a specimen; exposing the resulting mixture to predetermined conditions where a stimuli-responsive polymer aggregates; then measuring the turbidity of the mixture or a parameter correlated therewith; and calculating the amount of a detection target in a specimen based on a correlation equation for relating the amount of the detection target with the turbidity or the parameter under the predetermined conditions. The first half of the procedures is similar to the detection method as described above, and thus description thereof will be omitted.

Correlation Equation

A correlation equation for relating the amount of a detection target with the turbidity or a parameter correlated therewith is created under the same conditions as the aforementioned predetermined conditions. A more reliable correlation equation can be obtained when a larger amount of data is available from measurements of the amount of a detection target and the turbidity or a parameter for the correlation equation. Here, the above data may include 2 or more pieces of information about the amount of a detection target, preferably 3 or more pieces of information about the amount of a detection target.

Here, a correlation equation between the amount of a detection target and the turbidity may be either an equation directly correlating the amount of the detection target with the turbidity or a correlation equation between the amount of the detection target and a parameter reflecting the turbidity.

Calculation

A measured value of the turbidity of a mixture may be substituted into a correlation equation created as described above to calculate the amount of a detection target in a specimen.

There is no particular limitation for the parameter correlated with the turbidity, but it may be the strength of a signal upon being developed with a developing carrier (thin layer chromatography); the strength of a magnetic field when the first substance includes a magnetic substance; and the like.

A quantification method based on a signal upon being developed with a developing carrier is disclosed in WO2010/137532. Specifically, the method includes the steps of: measuring the strength of a signal resulting from the presence of a first conjugate or a second conjugate in the developing carrier; and calculating the amount of a detection target in a specimen based on a correlation equation for relating the amount of the detection target with the strength of a signal under predetermined conditions. The above method uses a phenomenon in which an aggregated stimuli-responsive substance will slowly develop in an appropriately selected developing carrier.

A detection method based on the strength of a magnetic field is disclosed in WO2009/084596. Specifically, the method includes the steps of: exposing a mixture under predetermined conditions where a stimuli-responsive polymer aggregates; then applying magnetic force to measure a magnetic field arising therefrom; and calculating the amount of a detection target in a specimen based on a correlation equation for relating the amount of the detection target with the strength of a magnetic field under the predetermined conditions. The above method uses a phenomenon in which aggregates will create a larger magnetic field.

Separation

When the first substance includes a particulate magnetic substance, the detection method or the quantification method according to embodiments of the present invention are preferred to further include applying magnetic force to separate the magnetic substance which undergoes aggregation. This enables the aggregated magnetic substance to be separated from impurities including the magnetic substance in an unaggregated state. As a result, the influence of impurities will be removed from measured values such as the amount of the separated magnetic substance and light transmittance when dispersed in a solvent. This will more accurately reflect the presence of a substance to be detected.

Application of magnetic force may be achieved by moving a magnet toward a magnetic substance. The magnetic force of the magnet may vary depending on the strength of the magnetic force of the magnetic substance used. Magnets include, for example, a Magna neodymium magnet.

Application of magnetic force may be performed before the determination or simultaneously in parallel with the determination, but the parallel determination is preferred in view of a shortened processing time. It is noted that an aggregated magnetic substance separated upon applying magnetic force may still contain impurities. This suggests that the turbidity of a mixture after the separation may rather be smaller in a case where impurities are present.

It is noted that the term “turbidimetry” in the detection method or the quantification method encompasses not only directly measuring turbidity but also measuring a parameter reflecting turbidity. Such parameters include the difference in measured values of turbidity at multiple time points, the amount of separated aggregates, the turbidity of an unaggregated substance after separation, and the like. Here, one of the multiple time points is preferably, for example, near a time point when the turbidity reaches the maximum value after applying magnetic force to a negative control having no detection target. This can ensure a larger difference with a measured value of turbidity at a different time point, allowing the amount of a detection target to be qualified more correctly.

Detection Target

Examples of the detection target in a specimen include substances for use in clinical diagnosis. Specifically, they include human immunoglobulins G, M, A and E, human albumin, human fibrinogen (fibrins and degradation products thereof), α-fetoprotein (AFP), C-reactive protein (CRP), myoglobin, carcinoembryonic antigens, hepatitis virus antigens, human chorionic gonadotropin (hCG), human placental lactogen (HPL), HIV virus antigens, allergens, bacterial toxins, bacterial antigens, enzymes, hormones (for example, human thyroid stimulating hormone (TSH), insulin, and the like), drug agents, and the like contained in body fluid, urine, sputum, feces, and the like.

Kit

The present invention also encompasses a kit for detecting and/or quantifying a detection target. The above kit includes first conjugates each in which a first substance including a stimuli-responsive substance is bound to a first affinity substance having an affinity for the detection target; and second conjugates each in which a second substance having a hydrophilic or electrically-charged moiety is bound to a second affinity substance having an affinity for the detection target, the second substances including particles, 0.01 or more of the particles having a specific gravity of 1.4 or more. The first substance preferably includes a particulate magnetic substance. The second substance is preferably configured such that a water soluble substance is attached on the surface of a high-specific gravity particle. The details of each component are as described above, and descriptions thereof are thus omitted.

### EXAMPLES

### Reference Examples 1 to 2 Measuring Recovery Rate of Unmodified Silica Particles

A colloidal solution in which unmodified silica particles shown in Table 1 were dispersed in water was prepared. The colloidal solution was then dispensed in an amount to give a dry weight before centrifugation as specified in 1) below using a Pipetman capable of dispensing 1 mg. Then the dry weight was measured in accordance with the methods of 1) to 3).

1) Before Centrifugation

The dispensed colloidal solution was dried in an incubator at 60° C. for 24 hours, and the dry weight of the silica particles was then measured.

2) 80 Minutes after Centrifugation

Centrifugation was performed at a gravitational acceleration of 20000 g for 80 minutes with a centrifuge, and the supernatant was then removed. Subsequently, the remaining pellet was dried in an incubator at 60° C. for 24 hours, and the dry weight of the silica particles was then measured.

3) 160 Minutes after Centrifugation

Centrifugation was performed at a gravitational acceleration of 20000 g for 160 minutes with a centrifuge, and the supernatant was then removed. Subsequently, the dry weight of the silica particles was measured as in the above 2). Results are shown in Table 1.

### Comparative Preparation Example 1 Preparation of Antibody-Modified Latex Particles

An aqueous dispersion of polystyrene latex particles (mean particle diameter: 50 nm) in an amount of 0.4 mL was mixed with anti-human IgG antibody (Medical & Biological Laboratories Co., Ltd.), and stirred with a stirrer at room temperature for 60 minutes to allow for physical adsorption of the anti-human IgG antibody to the latex particles. This reaction liquid was centrifuged at 20000 g for 80 minutes, and the supernatant was then removed. A PBS buffer (pH 7.4) was added to disperse the remaining pellet, and again centrifuged at 20000 g for 80 minutes, and the supernatant was then removed. This was dispersed in a PBS buffer (pH 7.4) containing 0.5% (w/v) BSA (Sigma), 0.5% (w/v) Tween® 20, and 10 mM EDTA to prepare a 0.025% dispersed solution of a second conjugate including anti-human IgG antibody-conjugated latex particles.

### Comparative Reference Examples 1 to 2 Measuring Recovery Rate of Latex Particles

A dispersed solution (Comparative Reference Example 1) of the antibody-modified latex particles obtained from Comparative Preparation Example 1 or a colloidal solution (Comparative Reference Example 2) in which the unmodified latex particles shown in Table 1 were dispersed in water was used to measure a dry weight in accordance with the methods of 1) to 3) as in Reference Examples 1 to 2.

TABLE 1

| | | | | Recovery of particles | |
|---|---|---|---|---|---|
| | | | | after being centrifuged | |
| | | | | at 20000 g | |
| | | | Content of | 80 | 160 |
| | Particles | Items | particles | minutes | minutes |
| Comparative | Latex(50 nm) + | Weight (mg) | 1.19 | 0.36 | 0.54 |
| Reference | Antibody(200 μg) | Recovery rate | — | 30% | 45% |
| Example 1 | | | | | |
| Comparative | Latex | Weight (mg) | 0.94 | 0.05 | 0.12 |
| Reference | particles(50 nm) | Recovery rate | — | 5% | 13% |
| Example 2 | | | | | |
| Reference | Silica | Weight (mg) | 1.08 | 0.85 | 1.13 |
| Example 1 | particles(50 nm) | Recovery rate | — | 79% | 105% |
| Reference | Silica | Weight (mg) | 1.01 | 0.62 | 0.95 |
| Example 2 | particles(30 nm) | Recovery rate | — | 61% | 94% |

As clearly shown in Table 1, the recovery rate of the unmodified latex particles was as low as about 10% after being centrifuged for 160 minutes even when the unmodified latex particles have a mean particle diameter of as large as 50 nm. The antibody-modified latex particles showed a higher recovery rate than the unmodified latex particles. In contrast, the silica particles, either of those having a mean particle diameter of 50 nm or 30 nm, showed much higher recovery rates than the antibody-modified latex particles.

### Example. Preparation of First Conjugate

First, an antibody (clone: Mouse 195, mouse IgG, Leinco Technology, Inc.), which was a first affinity substance, against human thyroid stimulating hormone (TSH), which was a detection target, was biotinylated by the well-known conventional sulfo-NHS-biotin method (AGC Techno Glass Co., Ltd.) to prepare a biotin-labeled anti-TSH beta antibody.

Meanwhile, 250 μL of Magnabeat Therma-Max LSA Streptavidin (0.4 mass %) as a streptavidin-attached particulate magnetic substance was transferred into a 1.5 mL micro tube. The Therma-Max LSA Streptavidin was allowed to aggregate by heating the micro tube to 42° C., and then magnetically collected. The supernatant was then removed. To this, added was 250 μL of a TBS buffer (20 mM Tris-HCl, 150 mM NaCl, pH 7.5). Aggregates were then dispersed by cooling. To this dispersed liquid, added was 50 μL of the biotin-labeled anti-TSH β antibody (0.75 mg/mL) dissolved in a PBS buffer (0.01 M phosphate buffer, 0.0027 M potassium chloride, 0.137 M sodium chloride, pH 7.4). This was subjected to upside-down mixing at room temperature for 15 minutes. The Therma-Max LSA Streptavidin was allowed to aggregate by heating the micro tube to 42° C., and then magnetically collected. The supernatant was then removed, and excessive biotin-labeled anti-TSH beta antibody was separated (B/F separation). To this, added was 250 μL of the TBS buffer. Aggregates were then dispersed by cooling. Subsequently, the excessive amount of biotin was added to cover the biotin binding sites of the streptavidin, and then excess biotin was separated (B/F separation). This was further dispersed in a PBS buffer (pH 7.4) solution containing 0.5% (w/v) BSA (Sigma), 0.5% (w/v) Tween® 20, and 10 mM EDTA to obtain a first conjugate.

Preparation of Second Conjugate

An aqueous dispersion of silica particles (specific gravity: 1.8, and mean particle diameter: 100 nm) in an amount of 0.4 mL was mixed with an antibody (clone: Mouse 195, mouse IgG, Leinco Technology, Inc.), which was a second affinity substance, having an affinity for human thyroid stimulating hormone (TSH), which was a detection target, and stirred with a stirrer at room temperature for 60 minutes to allow for physical adsorption of the anti-human IgG antibody to the silica particles. This reaction liquid was centrifuged at 20000 g for 80 minutes, and the supernatant was then removed. A PBS buffer (pH 7.4) was added to disperse the remaining pellet, and again centrifuged at 20000 g for 80 minutes. Then the supernatant was removed. This was dispersed in a PBS buffer (pH 7.4) containing 0.5% (w/v) BSA (Sigma), 0.5% (w/v) Tween® 20, and 10 mM EDTA to prepare a 0.025% dispersed solution of a second conjugate including anti-human IgG antibody-conjugated silica particles.

Sample Preparation

Human thyroid stimulating hormone (TSH; Aspen Bio Pharma, Inc., activity: 8.5 IU/mg, WHO80/558) as an antigen which was the detection target was dissolved in a PBS buffer (pH 7.4) to give a concentration of 30 μg/mL. This solution was diluted to 10 pg/mL, 50 pg/mL, 250 pg/mL, 500 pg/mL, 1000 pg/mL with a VITROS TSH calibrator 1 (TSH: 0 mIU/L, Ortho Clinical Diagnostics) to obtain samples.

Mixing

The first conjugate in an amount of 150 μL and the second conjugate in an amount of 120 μL were poured into a micro tube, and agitated with a vortex mixer for 1 second. To this micro tube, 750 μL of each sample was added, and again agitated with a vortex mixer for 60 seconds to obtain a liquid mixture. Similarly, a liquid mixture in which human thyroid stimulating hormone was not present was also prepared as a negative control.

Creation of Correlation Equation

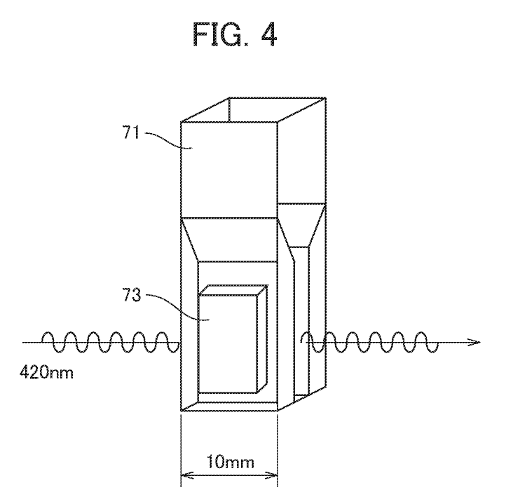

As shown in FIG. 4, a neodymium permanent magnet 73 with dimensions of 5 mm×9 mm×2 mm (Seiko Sangyo Co., Ltd.) was attached outside the optical path of a common semi-microcell 71 for a spectrophotometer. The cell 71 was placed in a visible-ultraviolet spectrophotometer “UV-3101PC” (Shimadzu Corporation) equipped with a cell-temperature controller, and was held at 32° C. for 10 minutes or longer.

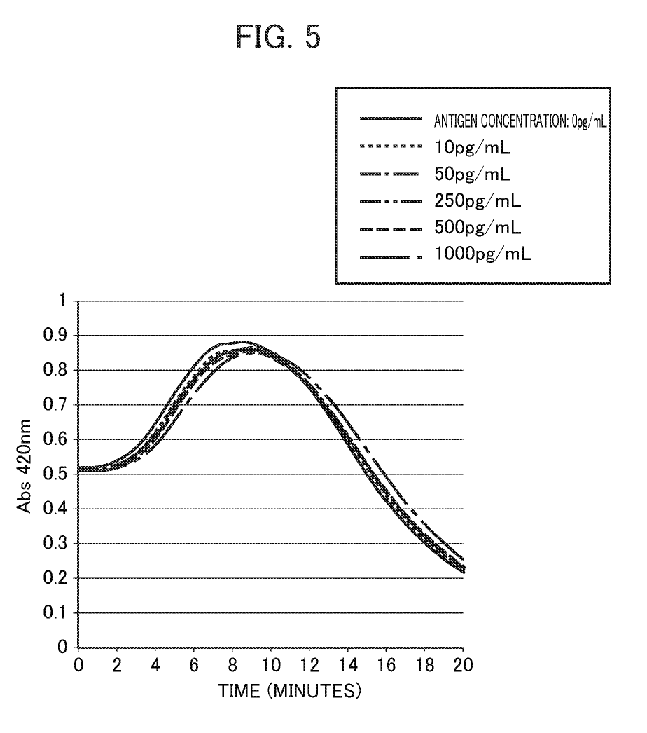

The above liquid mixture was dispensed into the cell, and zero-correction was performed according to the instructions of the spectrophotometer. Then turbidity (absorbance Abs) was directly and continuously measured over 20 minutes with a slit width of 10 mm using light having a wavelength of 420 nm. Similarly, turbidity (absorbance Abs) was also measured for the liquid mixture prepared as a negative control. Results are shown in FIG. 5.

The results shown in FIG. 5 demonstrate that the stimuli-responsive substance experiences more aggregation inhibition and remains more dispersed to show lower turbidity until about 9 minutes as the concentration of antigen as a detection target increases. However, the relationship between the concentration of antigen and the turbidity begins to be reversed, and the turbidity becomes decreased below the initial value over time. This likely occurs because the magnetic substance aggregated is adsorbed to the magnet, and then separated.

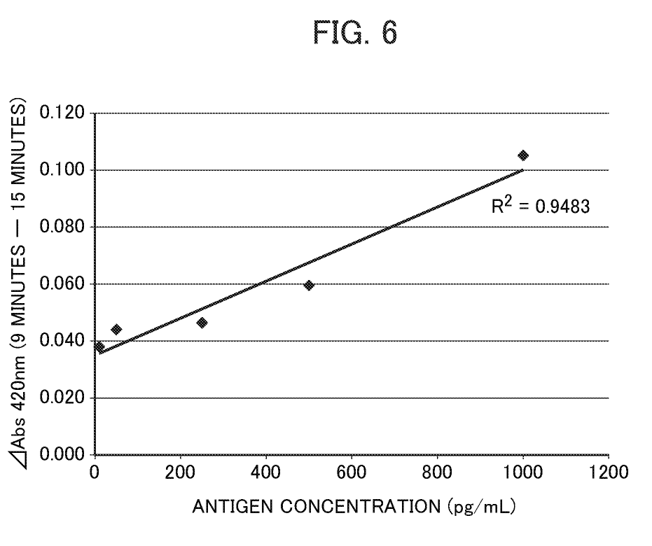

Next, a correlation equation is shown in FIG. 6, which relates the concentration of antigen in each sample with a value obtained by subtracting a first value of turbidity from a second value of turbidity, the first value being obtained by subtracting a measured value of turbidity at 15 minutes from a measured value of turbidity at 9 minutes for each antigen concentration, the second value obtained by subtracting a measured value of turbidity at 15 minutes from a measured value of turbidity at 9 minutes for an antigen concentration of 0. As shown in FIG. 6, a correlation equation was able to be obtained which had a correlation coefficient R² of as very high as 0.9483. Results showed that the above correlation equation was able to quantify the concentration of an antigen with a high degree of accuracy.

### EXPLANATION OF REFERENCE NUMERALS

10, 10A First conjugate
11 Stimuli-responsive substance
13 First antibody (First affinity substance)
13A Autoantigen (for example, CCP; first affinity substance)
15 Avidin
17 Biotin
19 Magnetic substance
20 Second conjugate
21 Second substance
23 Second antibody (Second affinity substance)
50, 50A Detection target

1. A method of detecting a detection target in a specimen, the method comprising:
mixing a first conjugate in which a first substance comprising a stimuli-responsive substance is bound to a first affinity substance having an affinity for the detection target, a second conjugate in which a second substance having a hydrophilic or electrically-charged moiety is bound to a second affinity substance having an affinity for the detection target, and the specimen; exposing the resulting mixture to conditions where the stimuli-responsive substance aggregates; and determining whether dispersion of the stimuli-responsive substance or an event having a correlation therewith is present,
the second substance comprising a particle having a specific gravity of 1.4 or more,
the first affinity substance and the second affinity substance being capable of simultaneously binding to the detection target at different sites of the detection target.

2. A method of quantifying a detection target in a specimen, the method comprising:
mixing a first conjugate in which a first substance comprising a stimuli-responsive substance is bound to a first affinity substance having an affinity for the detection target, a second conjugate in which a second substance having a hydrophilic or electrically-charged moiety is bound to a second affinity substance having an affinity for the detection target, and the specimen; exposing the resulting mixture to predetermined conditions where the stimuli-responsive substance aggregates;
measuring the turbidity of the mixture or a parameter having a correlation therewith; and calculating the amount of the detection target in the specimen based on a correlation equation for relating the amount of the detection target with the turbidity or the parameter under the predetermined conditions,
the second substance comprising a particle having a specific gravity of 1.4 or more,
the first affinity substance and the second affinity substance being capable of simultaneously binding to the detection target at different sites of the detection target.

3. The method according to claim 1, wherein the first substance includes a particulate magnetic substance, and
the method further comprises applying magnetic force to the mixture after exposing the mixture to the conditions to separate the magnetic substance in an aggregated state.

4. The method according to claim 1, wherein the second substance comprises a water soluble substance being attached to a surface of the particle.

5. A method of preparing a reagent for use in the method according to claim 1, the reagent comprising the second conjugate, the method comprising:
allowing the second substance to bind with the second affinity substance in water, and then performing solid-liquid separation to recover the second conjugate in a solid phase.

6. A kit for detecting and/or quantifying a detection target, the kit comprising:
a first conjugate in which a first substance comprising a stimuli-responsive substance is bound to a first affinity substance having an affinity for the detection target, and a second conjugate in which a second substance having a hydrophilic or electrically-charged moiety is bound to a second affinity substance having an affinity for the detection target,
the second substance comprising a particle having a specific gravity of 1.4 or more.

7. The kit according to claim 6, wherein the first substance includes a particulate magnetic substance.

8. The kit according to claim 6, wherein the second substance comprises a water soluble substance being attached to a surface of the particle.

9. The method according to claim 2, wherein the first substance includes a particulate magnetic substance, and
the method further comprises applying magnetic force to the mixture after exposing the mixture to the conditions to separate the magnetic substance in an aggregated state.

10. The method according to claim 2, wherein the second substance comprises a water soluble substance being attached to a surface of the particle.

11. A method of preparing a reagent for use in the method according to claim 2, the reagent comprising the second conjugate, the method comprising:
allowing the second substance to bind with the second affinity substance in water, and then performing solid-liquid separation to recover the second conjugate in a solid phase.

12. The method according to claim 1, the method further comprising:
allowing the second substance to bind with the second affinity substance in water, and then performing solid-liquid separation to recover the second conjugate in a solid phase.

13. The method according to claim 2, the method further comprising:
allowing the second substance to bind with the second affinity substance in water, and then performing solid-liquid separation to recover the second conjugate in a solid phase.

14. The method according to claim 1, wherein the first conjugate and the second conjugate is comprised in a kit.

15. The method according to claim 2, wherein the first conjugate and the second conjugate is comprised in a kit.
